(19) United States
(12) Patent Application Publication
(10) Pub. No.: US 2005/0283159 A1
(43) Pub. Date: Dec. 22, 2005

(54) Intramedullary osteosynthesis implant

(76) Inventor: Bouali Amara, Montpellier (FR)

Correspondence Address: HESLIN ROTHENBERG FARLEY & MESITI PC, 5 COLUMBIA CIRCLE, ALBANY, NY, 12203, US

(21) Appl. No.: 10/870,691

(22) Filed: Jun. 17, 2004

(51) Int. Cl. A61B 17/86

(52) U.S. Cl. 606/75; 606/72

(57) ABSTRACT

Intramedullary osteosynthesis implant, permitting in particular arthrodesis (1) of a joint, for example an interphalangeal joint, or diaphyseal osteosynthesis of the upper limb or of the lower limb, comprising two sets of at least two rods (2, 3, 4, 5) each extending on either side of a central zone (6), said rods (2, 3; 4, 5) being substantially parallel at ambient temperature within the same set, each set of rods being intended to be impacted in the medullary canal of a diaphysis, said implant being made from a shape-memory material so that, at body temperature, the rods (2, 3; 4, 5) of the same set spread apart so as to be able to immobilize themselves in said medullary canal.

### TECHNICAL FIELD

The invention concerns the field of surgical treatment of articular lesions with arthritis and/or deformation of the joints of the upper limb and of the lower limb, the therapeutic objective being to immobilize the joint by osseous fusion.

It concerns more particularly an arthrodesis implant and a surgical method of treating degenerative or post-traumatic arthritis and articular deformations by fitting such an implant.

The implant is also used as a means of intramedullary osteosynthesis when osseous fusion is sought in trauma surgery or corrective surgery.

In this application, the object is to obtain fusion and osseous consolidation of a diaphyseal or articular bone by means of an intramedullary implant.

### PRIOR ART

Arthritis of the interphalangeal joints, whether of the foot or of the hand, is generally quite common, considering that it affects about 20% of the population over 55 years of age and 40% over 70 years of age.

In practice, this arthritis is manifested by pain particularly when the patient makes a gripping movement between the thumb and index or middle finger, which are the fingers most subjected to stress. This arthritis causes various types of deformation, for example the presence of Heberden's nodes, or angulation or rotation between the phalanges. These deformations cause difficulties in gripping or result in instabilities of the joint which cause problems if executing movements requiring a certain precision or adequate force.

In practice, when the arthritis is relatively mild, whether in terms of intensity of pain or degree of deformation, incipient forms may be treated adequately by administration of anti-inflammatories, or even by injection of corticosteroids. By contrast, for advanced forms, surgical treatment is most suitable. This consists of arthrodesis of the interphalangeal joint, which consists in suppressing the mobility of the joint by bringing about osseous fusion of the two phalanges concerned.

Various techniques have already been proposed for performing this arthrodesis.

Thus, osseous fusion generally necessitates cutting the bone at the opposing articular ends. Numerous cutting forms have already been proposed, in the form of tenons or chevrons, or else by using concave and convex shapes. However, plane cutting is the most used.

This osseous cutting is accompanied by placement of osteosynthesis fixtures which can be of different types.

Thus, osteosynthesis can be obtained using Kirschner pins, with or without steel wire hoops, or by Herbert screws.

These different osteosynthesis fixtures cause considerable inconvenience for the patient. This is because they generally require immobilization of the joint by means of a splint for several weeks, the time needed to obtain osseous consolidation. It is also necessary to perform secondary ablation of the material after a few weeks, which then entails a new intervention under local anesthesia with additional skin incisions.

In addition, the pins present under the pulp can cause pain, ulceration, or infection, even perforation of the skin. In any event, they cause considerable functional impairment in most cases because the patient no longer uses his finger for quite a long period of time.

In addition, in about 20% of cases, this arthrodesis causes major complications, among which osteitis, deep infection, or malposition of the two phalanges after osseous fusion. Other complications, albeit less important, are observed in about 16% of cases, among which there may be mentioned cutaneous necrosis, scar dysesthesia, cold intolerance, superficial infection, even stiffness of the proximal interphalangeal joint of the same finger.

A problem which the invention proposes to solve is that of eliminating the drawbacks due to the need to remove all or part of the osteosynthesis equipment.

Another problem which the invention seeks to solve is that of permitting particularly stable arthrodesis ensuring fusion of the joint in question. Another objective of the invention is to give the patient the possibility of reusing the treated finger as rapidly as possible in the immediate postoperative period without being inconvenienced by the osteosynthesis material, and while having particularly stable osteosynthesis. Another problem which the invention seeks to solve is that of the complications (skin ulceration in the area of the pulp, pulpar scarring, exclusion of the finger, etc.) which the existing osteosynthesis articles cause in the area of the ends of the fingers.

### DISCLOSURE OF THE INVENTION

The invention thus firstly concerns an intramedullary osteosynthesis implant for arthrodesis and diaphyseal osteosynthesis. Arthrodesis signifies the articular fusion of the joints accessible to intramedullary osteosynthesis of the upper and lower limbs, among which there may be mentioned the proximal and distal interphalangeal joints, the interphalangeal joint of the thumb, but also the metacarpo-phalangeal joints and the carpal and radiocarpal joints.

The invention also concerns the joints of the lower limb for treatment of arthritis, but also of deformations such as claw toes. Mention may be made in particular of treatment of the interphalangeal joints, the metatarso-phalangeal joints, and the tarsal and tibio-tarsal joints.

Intramedullary diaphyseal osteosynthesis signifies the use of the implant with the aim of obtaining diaphyseal osseous fusion in the context of traumatology (such as open diaphyseal fractures, articular destruction, replantation of the fingers) or corrective surgery (such as diaphyseal osteotomy or joint transfer).

In accordance with the invention, this implant comprises two sets of at least two rods, and in practice two sets of two or three rods, without however excluding a possibly greater number of rods per set. Within the same set, these rods are substantially in the same plane and parallel to one another at ambient temperature. Each set of rods extends on either side of a central zone. Each set of rods is intended to be impacted in the diaphyseal medullary canal. Said central zone is in the form of a transverse rod or branch (horizontal) which, when cold, is perpendicular to the rods to be impacted. This central zone ensures the rigidity in flexion and substantial anti-rotation stability of the set, while maintaining a capacity for being angled, by virtue of its rectangular cross section. The implant is made from a shape-memory material so that, at body temperature, the rods of the same set spread apart in such a way as to be able to immobilize themselves in the medullary canal.

In other words, in the version with two pairs of rods, the implant comprises four rods connected in pairs to form a general H-shaped configuration which, when the temperature rises, deforms to adopt a general X-shape. When the implant is at ambient temperature, the rods of the same pair are parallel, thus permitting easy fitting in holes made for this purpose in the articular metaphysis. When the implant is exposed to a higher temperature, and in particular body temperature, the rods spread apart and anchor the implant in the diaphysis.

In practice, at body temperature, the rods of the same pair advantageously spread apart in the same plane as that defined when they are parallel at ambient temperature.

In one alternative embodiment, the implant comprises two sets of three rods forming a hexapod implant. At ambient temperature, the three rods of the same set are substantially in the same plane and parallel to one another, forming a three-pronged fork or parallel trident. Each trident is intended to be impacted in the diaphyseal medullary canal and at body temperature the outer rods of the same pair spread apart and diverge while remaining in the same plane, while the central rod spreads apart in a plane substantially perpendicular to the plane of the outer rods. The three rods immobilize in the medullary canal against the inner wall of the diaphysis of the bone, forming a double tripod which ensures particularly stable osteosynthesis.

In practice, at ambient temperature, the planes defined by each set of rods advantageously form between them an angle of between 0° and 60°, making it possible to regulate the inclination or flexum of arthrodesis, that is to say the angulation between the bone structures (joints, diaphyses) once they are immobilized with respect to one another.

This angle of inclination or flexum, which thus corresponds to the angle defined between each pair of rods, varies depending on the type of arthrodesis or diaphyseal osteosynthesis to be performed.

In practice, to facilitate impaction of the implant, the ends of each rod can be beveled.

In one particular embodiment, the two pairs of rods can have different lengths, the pair of rods which are longer being intended to be impacted in the joint or the proximal diaphysis.

In practice, the shape-memory material used to obtain the characteristic deformation can be based on a titanium and nickel alloy, for example the alloy known by the name Nitinol®. More precisely, the alloy used comprises between 43 and 45% titanium, preferably between 44 and 44.6%, and between 55 and 57% nickel, preferably between 55.4% and 56%. The alloy which has given the best results comprises about 44.3% titanium and about 55.7% nickel. This alloy is relatively malleable when in a martensitic state, when the implant is in an H-shape. It then changes to a very resistant austenitic state when the implant adopts an X-shape. The deformation of the implant thus takes place between the temperature of 22° C., corresponding to the conditions of fitting the article, and the temperature of 37° C. where the material finishes its deformation and ensures the stability of the fitting.

The cross section of the implant according to the invention advantageously has a width/height ratio of greater than 1:8 when cold and of greater than 2:5 when hot.

In practice, the stress measured at the end of the rods of an implant of the order of 10 millimeters long varies from 1.5 newton at the temperature of 20° C. to a value of 13 newton at 37° C.

The invention thus permits intramedullary osteosynthesis with the aim of obtaining osseous fusion which is either articular (arthrodesis) or diaphyseal. The indications are the treatment of:
articular deformations requiring arthrodesis;
degenerative and post-traumatic arthritis;
diaphyseal osteosynthesis in traumatology and corrective surgery.

More precisely, the operating technique for fitting this implant comprises the following steps:
making an incision in the dorsal face of the joint;
freeing the proximal and distal ends of the joint or diaphyses of the joint, in particular by luxation of this joint, and thus releasing the lateral ligaments of the joint;
resecting the osseous ends of the joint to obtain surfaces which, when they are in contact, reproduce the angulation desired after bone consolidation:
   for arthrodesis, the angulation formed during the bone cuts is a function of the type of joint (by way of example, 10° for the distal interphalangeal joint)
   for intramedullary diaphyseal osteosynthesis, it is not generally necessary to have any angulation, and bone cuts are made parallel to one another.
drilling holes to receive the rods (two or three depending on the implant model) in the medullary canal on the proximal bone then on the distal bone with the aid of a suitable ancillary or with the aid of drill bits and an osteosynthesis motor. In the case of drilling several holes with the aid of drill bits, their spacing is ensured by virtue of a drill guide with apertures spaced apart by the desired distance between the holes. A guide pin introduced into one of the already drilled holes makes it possible to keep the drill guide correctly positioned.
breaking-in the osseous bridge in the area of the proximal bone with the aid of a chisel to a depth of 2 to 3 mm in order to accommodate the horizontal branch of the implant there.
impaction of the implant in the holes of the proximal bone, then impaction of the distal bone in the distal rods of the implant (two or three rods depending on the implant model).

A so-called “phantom” implant makes it possible to simulate the arthrodesis or diaphyseal osteosynthesis before fitting the shape-memory implant. This “phantom” makes it possible to correct any error in position of the implant, and in particular the errors of angulation in the frontal, sagittal and horizontal planes.

### BRIEF DESCRIPTION OF THE FIGURES

The invention and the advantages which derive therefrom will become clear from the following description which is given with reference to the attached figures, in which:

FIG. 1 is a plan view of an implant according to the invention, comprising two pairs of rods, and shown at ambient temperature.

FIG. 2 is a side view of the implant from FIG. 1.

FIG. 3 is a plan view of the implant from FIG. 1, shown at human body temperature.

FIG. 4 is a plan view of an implant according to an alternative embodiment of the invention, comprising two sets of three rods, shown at ambient temperature.

FIG. 5 is a side view of the implant from FIG. 4.

FIG. 6 is a plan view of the implant from FIG. 4, shown at human body temperature.

FIG. 7 is a side view of the implant from FIG. 4, shown at human body temperature.

FIGS. 8 through 16 are plan views and side views showing the operating technique for fitting the implant from FIG. 1, using the example of a distal interphalangeal joint.

FIGS. 17 and 18 are diagrammatic representations, in plan and side views, respectively, of a finger in which the implant according to the invention has been fitted, after osseous fusion of the phalanges.

### EMBODIMENTS OF THE INVENTION

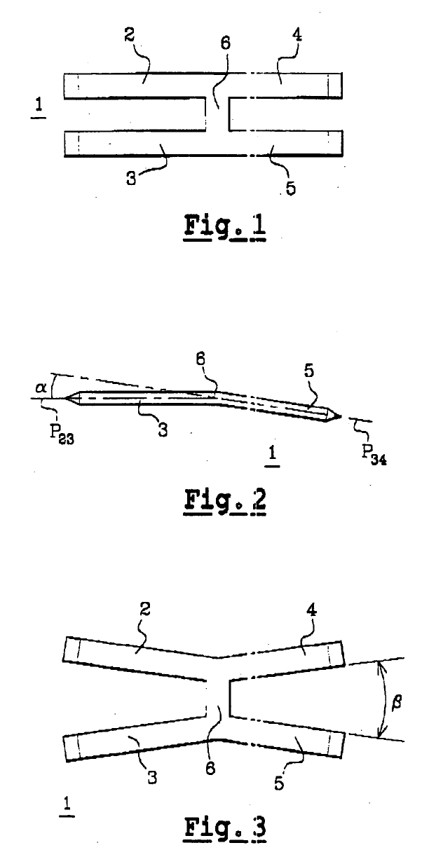

The arthrodesis implant (1) illustrated in FIG. 1 comprises two pairs of rods (2, 3, 4, 5) which are substantially parallel and are connected to a central portion (6) in such a way that the implant (1) has a general H-shape.

The implant illustrated in FIG. 1 is used especially for arthrodesis of the distal interphalangeal joint of the hands and the proximal interphalangeal joints of the toes. This implant has a total length of the order of 14 millimeters.

The total width of the implant (1), in the configuration at ambient temperature illustrated in FIG. 1, is of the order of 4.5 millimeters. Each rod has a width of the order of 1.1 millimeter. The rods of the same pair are separated by a space of the order of 4.5 millimeters.

In the form illustrated, the rods (2, 3) intended to be impacted in the joint or the proximal diaphysis are slightly longer than the rods (4, 5) intended to be impacted in the joint or the distal diaphysis. More precisely, in the example corresponding to the distal interphalangeal joint of the fingers, the longer rods (2, 3) have a length of the order of 8 millimeters, the shorter rods (4, 5) having a length of 6 millimeters.

As is illustrated in FIG. 2, each pair of rods (2, 3, 4, 5) is inscribed within a plane (P₂₃, P₄₅) . In the form illustrated, the planes (P₂₃, P₄₅) form between them an angle α of the order of 10°. This angle α corresponds substantially to the angle imposed on the phalanges connected by the implant and thus defines the flexum of arthrodesis. However, it will be noted that this angle α can vary depending on the type of joint of hand or foot on which arthrodesis is to be obtained. In practice, it can vary from 0° to 60°, and, for diaphyseal osteosyntheses, it is often 0°.

As is illustrated in FIG. 2, the ends of the rods (2, 3, 4, 5) are slightly beveled so as to permit good penetration of these into the proximal and distal impaction holes made in the articular bone surfaces after suitable cutting to the desired angle.

According to an important characteristic of the invention, the implant is made from a shape-memory material, as has been described above, allowing the rods (2, 3, 4, 5) to diverge when the temperature of the implant changes from ambient temperature, of the order of 20° C., to human body temperature, namely 37° C.

More precisely, this shape transition takes place starting from 22° C. and up to about 37° C.

The form illustrated in FIG. 3 shows the implant at a temperature of 37° C., the rods (2, 3) and (4, 5) between them forming an angle β of the order of 20° on a free implant without osseous constraint.

When the implant is in the bone, this angle varies as a function of the bone strength encountered in the metaphysis or in the diaphysis. The angle is open in an osteoporotic bone of low strength, whereas it is closed in a strong bone, because in this case the stresses applied against the osseous walls of the diaphysis are greater.

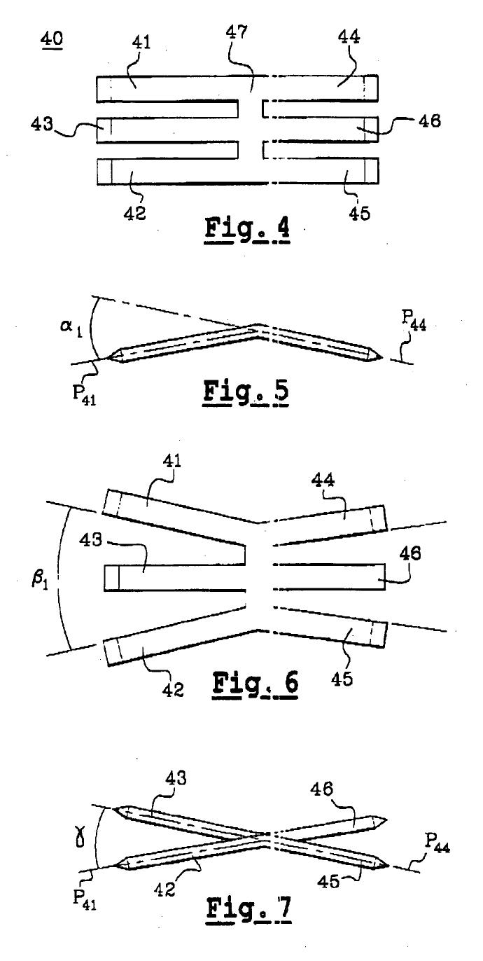

As has already been mentioned, the invention also covers implants of more complex shapes. Thus, the implant for arthrodesis or diaphyseal osteosynthesis (40) illustrated in FIG. 4 comprises two sets of three rods (41-46) which are substantially parallel and are connected to a central portion (47), in such a way that the implant (40) has a double trident shape.

The implant illustrated in FIG. 4 is used especially for arthrodesis of the “large” joints of the upper limb and of the lower limb, among which particular mention may be made of the proximal interphalangeal joints of the hands and of the toes, the metacarpo-phalangeal and metatarso-phalangeal joints. This implant (40) has a total length which can range from 17 mm for the proximal interphalangeal joints to about 33 mm for the metatarso-phalangeal joints.

The total width of the implant (40), in the configuration at ambient temperature illustrated in FIG. 4, is of the order of 5 millimeters. Each rod (41-46) has a width of the order of 1.5 to 2 millimeters. The rods of the same trio are separated by a space of the order of 1.5 millimeter.

In the form illustrated, the rods (41-43) intended to be impacted in the joint or the proximal diaphysis are slightly longer than the rods (44-46) intended to be impacted in the joint or the distal diaphysis. More precisely, in the example corresponding to the proximal interphalangeal joint of the fingers, the longer rods (41-43) have a length of the order of 10 mm, the shorter rods (44-46) having a length of 7 mm.

As is illustrated in FIG. 5, each set of rods (41-46) is inscribed in a plane (P₄₁, P₄₄). In the form illustrated, the planes (P₄₁, P₄₄) between them form an angle α1 which varies from 0° to 60° depending on the joint or diaphysis in question. This angle α1 corresponds substantially to the angle imposed on the phalanges or metacarpals or metatarsals connected by the implant and thus defines the flexum of arthrodesis. This angle α1 varies depending on the type of joint of hand and foot on which arthrodesis is to be obtained.

As has already been mentioned, the implant is made from a shape-memory material, as described above, which allows the outermost rods (41, 42; 44, 45) to diverge and spread apart in the planes (P₄₁, P₄₄) when the temperature of the implant changes from ambient temperature, of the order of 20° C., to the human body temperature of 37° C. At the same time, the central rods (43, 46) spread apart by displacement in a plane substantially perpendicular to the planes of displacement (P₄₁, P₄₄) of the outermost rods.

The form illustrated in FIG. 6 shows the implant at a temperature of 37° C., the outer rods (41, 42) and (44, 45) between them forming an angle β1 of the order of 20° on a free implant without osseous constraint.

The angle γ illustrated in FIG. 7 corresponds to the angle of divergence of the central rods (43, 46) in a vertical plane relative to the planes (P₄₁, P₄₄) of the outer rods (41, 42; 44, 45).

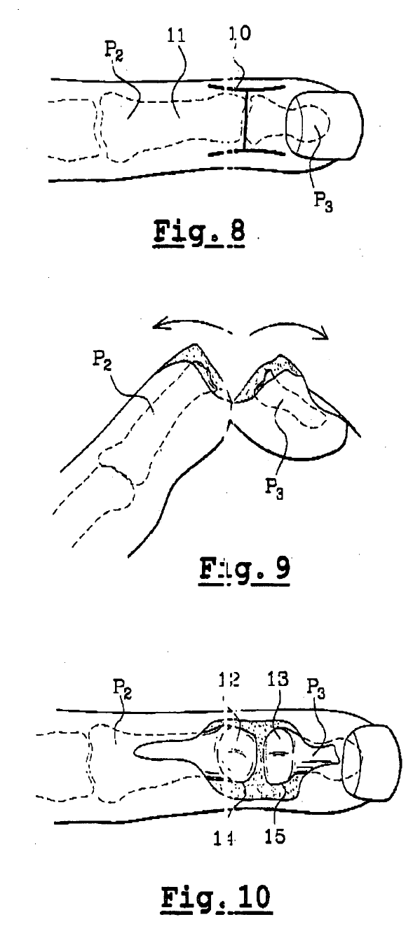

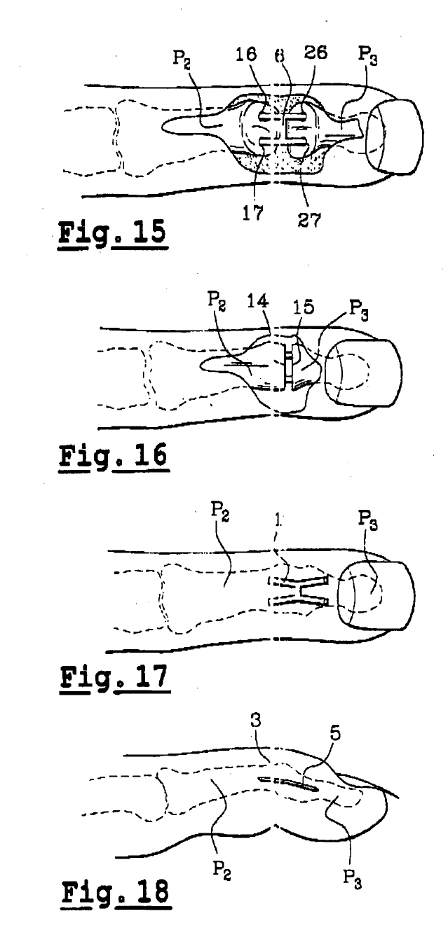

As has already been mentioned, the invention also concerns the method of fitting this implant in the area of an interphalangeal joint. In the example illustrated in FIGS. 8 and 15, the implant is fitted on a distal interphalangeal joint where the lateral bands of the extensor tendon apparatus are sectioned. However, it goes without saying that the operating technique for fitting the implant can be transposed without difficulty to other joints of the upper limb and of the lower limb, respecting the extensor tendon apparatus and, depending on the joint or diaphysis, the surrounding soft anatomical structures (vessels, nerves, tendons, etc.).

On joints more voluminous than the distal interphalangeal joint, hexapod implants are fitted, as illustrated in FIGS. 4 through 7, which ensure even greater stability of osteosynthesis, particularly in the case of the interphalangeal and metacarpo-phalangeal joints but also the joints of the wrist and of the foot.

The operating technique varies depending on the type of joint, and the example given below concerns arthrodesis of the distal interphalangeal joint of a finger, it being understood that the person skilled in the art will be easily able to derive from this the operating technique for other applications of the invention.

Thus, in order to fit the implant, it is necessary in the first instance to make an H-shaped incision (10), as is illustrated in FIG. 8, in the dorsal face (11) of the distal interphalangeal joint.

As is illustrated in FIG. 9, the following operation consists in freeing the head (12) of the median phalanx P₂ and the base (13) of the distal phalanx P₃. This release consists first in performing a luxation of the joint by freeing the lateral ligaments connecting the phalanges P₂ and P₃. In the palmar zone of the joint, the palmar plaque will be completely freed in its proximal part. After sectioning the lateral ligaments and cutting the bone at the base of the distal phalanx (P₃) and the head of the median phalanx (P₂), a space between the two phalanges (P₂, P₃) greater than 6 mm is obtained after distraction. This space is intended to permit the passage of the distal rods of the implant and thus impaction of the distal phalanx (P₃) on the rods.

Thereafter, as is illustrated in FIG. 10, the osteophytes present on the ends of the two phalanges (P₂) and (P₃) are resected.

The bone of the head (12) of the median phalanx P₂ and of the base (13) of the distal phalanx (P₃) is then cut to form surfaces (14, 15) in very slight palmar inclination in order to reproduce the angle α of 10° of the implant when the latter is intended for a third finger (middle finger).

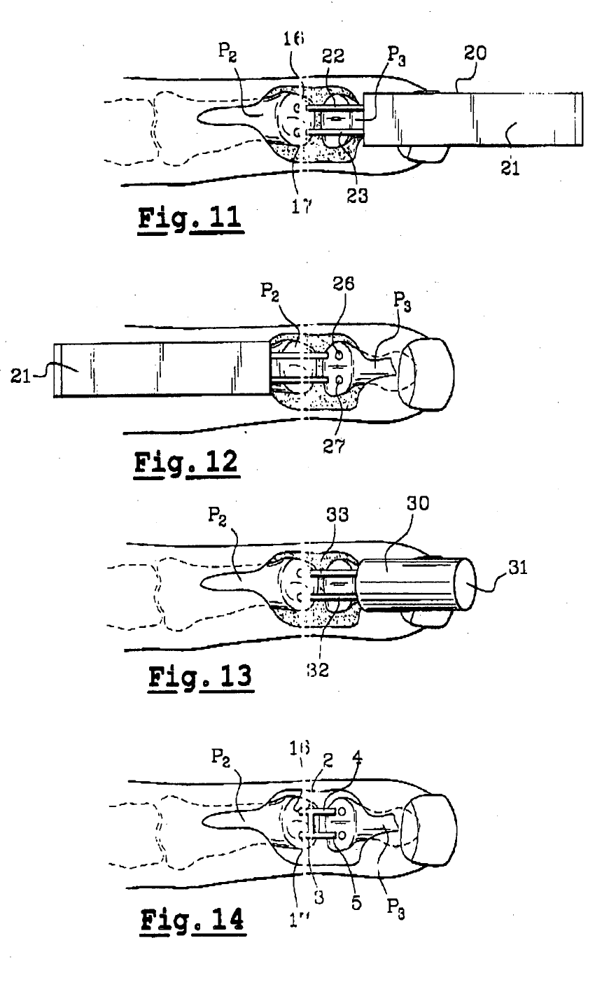

Thereafter, and as is illustrated in FIG. 11, holes (16, 17) are formed in the medullary canal of the median phalanx (P₂) . These holes (16, 17) are made with the aid of an ancillary (20) comprising a grip handle (21) continued by two parallel points (22, 23) having a spacing analogous to that of the rods (2, 3) of the implant (1). More precisely, after drilling with the aid of the ancillary (20), the formation of the holes (16, 17) may require the use of specific rasps for giving the holes (16, 17) the diameter corresponding to that of the rods (2, 3) of the implant (1).

Alternatively, these holes can be drilled with the aid of drill bits and an osteosynthesis motor. In the case of drilling several holes with the aid of drill bits, their spacing is ensured by means of a drill guide which has three apertures spaced apart by the desired distance between the holes. A guide pin introduced into one of the already drilled holes helps keep the drill guide correctly positioned.

Thereafter, the holes (26, 27) are formed in the same way in the medullary canal of the distal phalanx (P₃), as is illustrated in FIG. 12.

In order to ensure better osseous contact at the arthrodesis site, the osseous bridge in the area of the proximal bone is broken-in with the aid of a chisel to a depth of 2 to 3 mm. This notch in the osseous bridge makes it possible to accommodate the horizontal branch of the implant.

Before fitting the final implant, a “phantom” implant will make it possible to verify the correct position of the holes and thus the absence of angulation (in a horizontal plane) or of rotation (in a frontal plane) in the area of the future arthrodesis. It is thus possible to verify the correct convergence of the scaphoid upon closure of the fingers and the functional aspect (satisfactory flexum) and final esthetic aspect of the finger before fitting the final implant.

The final implant is protected in two cylinders, a long proximal cylinder and a short distal cylinder, in which the rods are encased. These cylinders protect the rods to keep them parallel to one another during their conditioning.

Thereafter, and as is illustrated in FIGS. 13 and 14, the long proximal rods (32, 33) are positioned opposite the holes (16, 17) formed on the median phalanx P₂. One of the cylinders forming an impactor (30) to facilitate manipulation of the implant will make it possible to introduce the latter into the holes (16, 17) of the median phalanx. The horizontal branch of the implant is also impacted in P₂, in the area of the notch formed in the osseous bridge. Thereafter, as is illustrated in FIG. 15, the implant (1) is finally fitted, ensuring impaction of the distal phalanx (P₃) on the distal pair of rods (4, 5) of the implant.

As is illustrated in FIG. 16, the plane walls of the median phalanx P₂ and distal phalanx P₃ come into contact with one another. It is necessary to ensure very good bone contact without any free space in the area of arthrodesis.

This is followed by final verification of the correct orientation of the finger and of the compressive effect on the arthrodesis site by impacting the distal phalanx (P₃) on the median phalanx (P₂) before proceeding to suture the cutaneous surfaces originally incised. The tourniquet is then loosened, which brings about the increase in temperature. The implant then deforms and is stressed against the diaphyseal walls of the medullary canal, thereby ensuring very stable osteosynthesis.

The operating technique employed when using hexapod implants as illustrated in FIGS. 4 through 7 varies depending on the type of joint or diaphysis treated. As regards more “voluminous” joints of the upper limb or lower limb, namely the proximal interphalangeal joint, metacarpo-phalangeal joint, metatarso-phalangeal joint, etc., it is important to access the site in a way which preserves the extensor apparatus by making incisions in the axis of the tendon, without disinsertion, so as to preserve the tendinous function, especially for the more distal joint(s).

Various trials were conducted on twelve different patients, for eighteen separate interventions, concerning the interphalangeal joints of the index finger, middle finger and little finger. More precisely, six interventions were performed on the index finger, five on the middle finger, and seven on the little finger.

Among the patients, ten were female and two male, with an average age of 56 years, ranging between 39 and 75 years. Seven of the twelve patients were of working age, and of these seven there were three who carried out manual work, three others employed as secretaries, and one a musician.

In 80% of the cases, the operations were performed on the dominant hand.

Among these twelve patients, pre-operative assessment revealed pain at rest in 58% of the cases, and pain on mobiliization in 83% of cases. In 83% of the cases, these pains were debilitating, and in 58% of the cases they manifested themselves upon temperature variation. In 41% of the cases, treatment of the analgesic type had been prescribed.

In 83% of the cases, the deformation of the distal interphalangeal joint was considered an impediment. In 67% of the cases, the mobility of the joint was considered as being severely diminished, and in the remaining 33% of the cases as being slightly diminished.

After an intervention in accordance with the invention, an evaluation of the complications was carried out.

Among the complications considered as minor, dysesthesia in the area of the scar was observed in 25% of the cases, and intolerance to cold in 25%. By contrast, there was no cutaneous necrosis, superficial cutaneous infection, nail dystrophy, or stiffness of the proximal interphalangeal joint.

Among the complications considered as major, no osteoarthritis of the distal interphalangeal joint was observed, and there was no case of malposition, either in rotation or angulation.

One case of pseudarthrbsis and of algodystrophy was identified in the same female patient after six months, but this complication finally disappeared at the end of 6 months when checked by radiography.

In terms of function, as from the immediate post-operative period (24 to 48 hours) the patients were able to use their hand normally in flexion/extension of the fingers, but without exerting stress at the site of arthrodesis. After the fourth week, but in some cases after up to 6 months, osseous fusion had taken place (osseous consolidation on radiology). The osteosynthesis material being purely intramedullary without exteriorization to the skin, it was possible to use the hand from the day after the operation.

FIGS. 17 and 18 correspond to illustrations made on the basis of radiography images performed between the second and sixth months, showing substantial osseous fusion with entirely satisfactory stability.

It will be apparent from the foregoing that the implant according to the invention and the surgical method for treating arthrosis by arthrodesis and intramedullary diaphyseal osteosynthesis has numerous advantages, particularly that of permitting stable osteosynthesis from the immediate postoperative period, without pulpar access or material, compared in particular with the osteosynthesis pins used hitherto. By virtue of stable osteosynthesis, the implant fitted according to the invention permits early mobilization and rapid use of the hand, without a splint, in the immediate postoperative period and without the sometimes considerably long periods needed for osseous fusion confirmed by radiography. This osseous fusion, whatever the technique, can take months to develop on account of the stability of the osteosynthesis. The intramedullary osteosynthesis implant according to the invention means that it is not necessary to wait for this radiological osseous fusion.

In addition, the surgical technique for fitting the implant is simple, reproducible and quick. In the majority of cases, an ancillary device makes it possible to do without osteosynthesis motors and thus avoid the economic costs of sterilizing and conditioning of an osteosynthesis motor.

1. An intramedullary osteosynthesis implant, permitting in particular arthrodesis (1) of a joint, or diaphyseal osteosynthesis of the upper limb or of the lower limb, comprising two sets of at least two rods (2, 3, 4, 5) each extending on either side of a central zone (6), said rods being substantially parallel at ambient temperature within the same set, each set of rods (2, 3; 4, 5) being intended to be impacted in the medullary canal of a diaphysis, said implant being made from a shape-memory material so that, at body temperature, the rods (2, 3; 4, 5) of the same set spread apart so as to be able to immobilize themselves in said medullary canal.

2. The implant as claimed in claim 1, comprising two sets of two rods, wherein, at body temperature, the rods (2, 3; 4, 5) of the same set spread apart in the same plane.

3. The implant as claimed in claim 2, wherein, at ambient temperature, the planes (P₂₃, P₄₅) defined by each set of rods (2, 3; 4, 5) between them form an angle (α) of between 0° and 60°.

4. The implant as claimed in claim 3, wherein the angle (α) between the planes (P₂₃, P₄₅) defined by each pair of rods is about 10°.

5. The implant (40) as claimed in claim 1, comprising two sets of three rods (41-46), wherein, at body temperature, the outer rods (41, 42; 44, 45) of the same set spread apart in the same plane, the central rod (43, 46) spreading apart outside of said plane.

6. The implant as claimed in claim 1, wherein the ends of each rod (2, 3; 4, 5) are beveled.

7. The implant as claimed in claim 1, wherein the two sets (2, 3; 4, 5) of rods have two different lengths, the longer set of rods (2, 3) being intended to be impacted in the joint or the proximal diaphysis.

8. The implant as claimed in claim 1, wherein it is made of a titanium and nickel alloy.

9. The implant as claimed in claim 8, wherein the alloy comprises between 43 and 45% titanium and between 55 and 57% nickel.

10. The implant as claimed in claim 9, wherein the alloy comprises between 44 and 44.6% titanium and between 55.4 and 56% nickel.

11. The implant as claimed in claim 1, wherein its cross section has a width to height ratio of greater than 1:8 when cold and of greater than 2:5 when hot.

12. A method of arthrodesis or intramedullary osteosynthesis using the implant as claimed in claim 1.

13. The method of arthrodesis or osteosynthesis as claimed in claim 12, comprising the following steps:
making an incision in the dorsal face of the joint;
freeing the proximal and distal ends of the joint;
resecting the osseous ends of the joint to obtain substantially plane surfaces;
drilling holes in the medullary canal at the proximal and distal ends;
successive impaction of the rods of the implant in the holes of the proximal and distal ends.

14. The method as claimed in claim 13, in which, after the holes have been drilled, the osseous bridge at the area of the proximal end is broken in, permitting impaction of the central zone of the implant.

15. The method as claimed in claim 13, used for arthrodesis of an interphalangeal joint.

16. The method as claimed in claim 15, used for arthrodesis of phalanges of the upper limb.

17. The method as claimed in claim 15, used for arthrodesis of phalanges of the lower limb.

18. The method as claimed in claim 15, used for arthrodesis of the distal and median phalanges of the upper limb or lower limb.

19. The method as claimed in claim 12 applied to the treatment of degenerative and post-traumatic arthritis, articular deformations, and diaphyseal fractures of the upper limb or lower limb.
